(19) United States
(12) Patent Application Publication
(10) Pub. No.: US 2009/0269849 A1
(43) Pub. Date: Oct. 29, 2009

(54) Bioreactor Apparatus

(75) Inventors: Chanyong Brian LEE, Newbury Park, CA (US); Joseph Gregory ZEIKUS, Okemus, MI (US)

Correspondence Address: ROTHWELL, FIGG, ERNST & MANBECK, P.C., 1425 K STREET, N.W., SUITE 800, WASHINGTON, DC, 20005, US

(73) Assignee: PBS Biotech, Inc., Newbury Park, CA (US)

(21) Appl. No.: 12/428,778

(22) Filed: Apr. 23, 2009

Related U.S. Application Data
(60) Provisional application No. 61/047,880, filed on Apr. 25, 2008.

(51) Int. Cl. C12N 5/00; C12M 1/00; C12N 1/00

(52) U.S. Cl. 435/383; 435/289.1; 435/243

(57) ABSTRACT

A bioreactor includes a fluid containment vessel and a rotating mixing element including radial inflow elements configured to direction fluid radially inwardly of the mixing element and axial out flow elements configured to redirect the radial inward flow in an axial direction. Both the vessel and the mixing element are made from plastic and the vessel comprises a flexible bag supported by a rigid housing. In one embodiment, the mixing element is pneumatically driven by the buoyancy of gas introduced into the vessel and trapped within gas entrapment cups formed on the periphery of the mixing element. As the volumetric capacity of the bioreactor increases, the ratio of the diameter of the mixing element to the width of the vessel decreases, and a gap defined between the bottom of the mixing element and the bottom of the vessel increase.

### PRIORITY CLAIM

This application claims the benefit under 35 U.S.C. 119(e) of U.S. Provisional Application Ser. No. 61/047,880, filed Apr. 25, 2008, the disclosure of which is hereby incorporated by reference.

### BACKGROUND

1. Field of the Invention

The field of the invention pertains to apparatuses for mixing solutions. More particularly, the invention relates to pneumatically operated mixers for use in closed, sterile environments.

2. Description of the Related Art

A cell culture or fermentation procedure embodies a technique for growing and proliferating unit cells separate from an organism and is widely used in biology, medical science, pharmacy, and agriculture. Additionally, the use of biological cultivation procedures has expanded into other disciplines, such as the treatment of waste water or oil.

Apparatuses designed for cultivation of microbial organisms or eukaryotic cells, known as bioreactors, have been used for production of various biological or chemical products in the pharmaceutical, biotechnological and beverage industry. A bioreactor includes a vessel for containing culture medium in a sterile environment that provides the various nutrients required to support growth of the homogeneous biological agents of interest.

Effective cell culture process requires appropriate supplies of nutrient substances, such as glutamine, glucose, and other medium components, and gas, such as oxygen, and carbon dioxide, for the growing cells in a bioreactor. In addition, timely control of physiological conditions such as appropriate pH, temperature, and osmolarity is required for mass cell culture production. In order to provide optimal culture conditions in a bioreactor, rapid and effective mixing in the culture medium is prerequisite, and cells should be uniformly dispersed throughout the culture medium without aggregation in any portion of the cultivation vessel.

Conventional bioreactors use mechanically driven impellers to mix the liquid medium during cultivation. Such bioreactors can be reused for the next batch of biological agents, but only after cleaning and sterilization of the vessel. The procedure of cleaning and sterilization requires a significant amount of time and resources, especially to monitor and to validate each cleaning step prior to reuse for production of biopharmaceutical products. Due to the high cost of construction, maintenance, and operation of the conventional bioreactors, single use bioreactor systems made of disposable plastic material have become an attractive alternative.

While several mixing methods in disposable bioreactors have been proposed in recent years, many of these methods work well only within certain bioreactor vessel size ranges. Problems sometimes arise, however, as larger mixing systems are attempted, and achieving efficient mixing for large scale (greater than 1000 liters) without expensive operating machinery has been a challenge. For this reason, a number of non-invasive and/or disposable mixing systems that do not require an external mechanical operation have been developed.

Various pneumatic bioreactors have been proposed, for example, in U.S. patent application Ser. Nos. 11/258,742 (Publication No. US 2007-0091716 A1); 11/739,089 (Publication No. US 2008-0261299 A1); and 11/739,659 (Publication No. US 2008-0268530 A1) (the respective disclosures of which are hereby incorporated by reference) assigned to the assignee of the present application. In this context, a “pneumatic” bioreactor is an apparatus having a vessel and a rotating mixing mechanism disposed within the vessel and which is actuated, or rotated, by means of the buoyant force generated by gas injected into the vessel and impinging on the mixing mechanism.

### SUMMARY

Aspects of the invention are embodied in a bioreactor comprising a vessel constructed and arranged to contain a fluid medium, at least one medium addition conduit configured for conveying liquid medium into the vessel, at least one medium removal conduit configured for conveying liquid medium from the vessel, a gas inlet conduit configured for introducing a gas into the vessel, and a mixing wheel mounted within the vessel for rotation about a substantially horizontal axis of rotation for mixing fluids within the vessel. The mixing wheel comprises a hollow cylinder, a plurality of gas entrapment elements positioned about the outer periphery of the cylinder and configured to capture at least a portion of the gas introduced into the vessel by the gas inlet conduit and cause the mixing wheel to rotate due to the buoyancy of the entrapped gas, and one or more deflector vanes disposed within an interior portion of the hollow cylinder and constructed and arranged to direct fluid within the hollow cylinder in a generally axial direction with respect to the cylinder.

According to another aspect, the bioreactor further comprises one or more radial inflow elements disposed at one or more locations about the periphery of the cylinder. Each radial inflow element is constructed and arranged to generate a radial fluid inflow into the interior portion of the cylinder when the mixing wheel is rotating about the axis of rotation. The deflector vanes are configured to redirect at least a portion of the radial fluid inflow in the generally axial direction with respect to the cylinder.

According to another aspect, the radial inflow elements comprise radial deflector vanes disposed across openings formed in the cylindrical wall.

According to another aspect, each deflector vane includes a leading edge portion extending beyond an axial end of the cylinder.

According to another aspect, each gas entrapment element comprises a partially enclosed cup having an opening facing in a circumferential direction with respect to the cylinder.

According to another aspect, the mixing element comprises a wheel with one or more flaps disposed about the periphery of the wheel, and wherein each flap is movable between a closed position lying along the circumference of the wheel and an opened position extending radially outwardly from the periphery of the wheel.

According to another aspect, each flap has a generally flat shape.

According to another aspect, the bioreactor further comprises a rotation measuring element constructed and arranged to measure a rate of rotation of the mixing element.

According to another aspect, the vessel comprises a flexible bag and wherein the bioreactor further comprises a rigid housing supporting the flexible bag.

According to another aspect, the flexible bag comprises generally parallel front and rear panels, generally parallel side panels, and a curved bottom panel, and the housing comprises a rigid curved panel conforming to the shape of the curved panel of the flexible bag.

According to another aspect, the curved bottom panel of the flexible bag has a generally semi-circular curvature curved about an axis that is parallel to the axis of rotation of the mixing element.

According to another aspect, the at least one medium addition conduit comprises one or more of a base addition conduit connected to a source of base material, an inoculum/seed addition conduit connected to a source of inoculum/seed material, and a nutrition feed medium addition conduit connected to a source of nutrient medium.

According to another aspect, a filter is provided on the gas inlet conduit.

According to another aspect, the bioreactor further comprises a heating element constructed and arranged to apply heat to the contents of the vessel.

According to another aspect, the bioreactor further comprises sensors for detecting one or more characteristics of the fluid medium contained within the vessel.

According to another aspect, the sensors comprise one or more of a temperature sensor, a dissolved oxygen sensor, and a pH sensor.

According to another aspect, the radial inflow element comprises a vane angled in a direction of rotation of the mixing element.

According to another aspect, the axial outflow element comprises deflector panels disposed radially inwardly of the outer periphery of the mixing element.

According to another aspect, the mixing element comprises a mixing wheel which comprises first and second radially oriented outer surfaces axially spaced from one another, each outer surface comprising an outer annular portion, a shaft ring located radially inwardly from the annular portion, and angularly-spaced spokes connecting the outer annular portion to the shaft ring. The mixing wheel further comprises a rotation shaft extending through the shaft rings of the first and second outer surfaces, an axially-oriented cylindrical wall extending between the first and second outer surfaces, the cylindrical wall including openings formed in different locations about the periphery thereof, a plurality of gas entrapment cups mounted on the cylindrical wall and extending about the periphery of the cylindrical wall, each of the gas entrapment cups being open in a circumferential direction with respect to the cylindrical wall, radial deflector vanes disposed across each of the openings formed in the cylindrical wall, and axial deflector panels extending from one spoke of the first outer surface to an angularly-spaced spoke of the second outer surface.

According to another aspect, the flexible bag is formed from polyethylene or polyvinylidene fluoride (PVDF).

According to another aspect, the mixing element is formed from plastic.

According to another aspect, the plastic comprises polyvinylidene fluoride (PVDF).

Other aspects of the invention are embodied in a bioreactor system comprising two or more bioreactor apparatuses of increasing volumetric capacity. Each bioreactor apparatus comprises a fluid containment vessel having a transverse dimension, at least one medium addition conduit configured for conveying liquid medium into the fluid containment vessel, at least one medium removal conduit configured for conveying liquid medium from the fluid containment vessel, and a mixing rotor rotatably disposed within the fluid containment vessel and having a transverse dimension. As the volumetric capacity of the bioreactor apparatuses increases, a ratio of the transverse dimension of the mixing rotor to the transverse dimension of the fluid containment vessel decreases.

According to another aspect, the fluid containment vessel of each bioreactor apparatus comprises a bottom panel, and the mixing rotor of each bioreactor apparatus is disposed within the fluid containment vessel to define a gap between a bottom portion of the mixing rotor and the bottom panel. As the volumetric capacity of the bioreactor apparatuses increases, the size of the gap increases.

Other aspects of the invention are embodied in a method for cultivating microbial organisms. The method comprises the steps of (A) adding an amount of culture medium to a fluid containment vessel having a transverse dimension, (B) adding materials to the culture medium to promote growth of the microbial organisms, (C) injecting a gas into the culture medium contained within the fluid containment vessel, and using the injected gas to rotate a mixing rotor rotatably disposed within the fluid containment vessel and having a transverse diameter. Next, in step (D) the culture medium is transferred to another fluid containment vessel, and the fluid containment vessel to which the culture medium is transferred has a larger volumetric capacity and larger transverse dimension than the fluid containment vessel from which the culture medium was transferred. After transferring the culture medium to a larger vessel, the method continues with the steps of (E) adding materials to the culture medium, including materials to promote growth of the microbial organisms, until the volumetric capacity of the fluid containment vessel to which the culture medium was transferred is reached, (F) injecting a gas into the culture medium contained within the fluid containment vessel, and using the injected gas to rotate a mixing rotor rotatably disposed within the fluid containment vessel to which the fluid medium was transferred. The mixing rotor has a transverse diameter, and a ratio of the transverse diameter of the mixing rotor to the transverse dimension the fluid containment vessel to which the culture medium was transferred is less than a ratio of the transverse diameter of the mixing rotor to the transverse dimension the fluid containment vessel from which the culture medium was transferred.

According to another aspect, steps (D)-(F) are repeated until a desired volume of culture medium having a desired concentration of microbial organisms is achieved.

These and other features, aspects, and advantages of the present invention will become apparent to those skilled in the art after considering the following detailed description, appended claims and accompanying drawings.

### BRIEF DESCRIPTION OF THE DRAWINGS

FIG. 1 is a perspective view of a bioreactor apparatus embodying aspects of the present invention, shown with the upper lid and front doors of the apparatus housing, which supports the disposable bioreactor vessel containing liquid medium, in their respective open positions.

FIG. 2 is an exploded perspective view of the bioreactor apparatus with the disposable bioreactor vessel shown outside of the housing.

FIG. 3 is a front plan view of the disposable bioreactor apparatus vessel.

FIG. 4 is an end plan view of the disposable vessel.

FIG. 5 is a perspective view of the disposable vessel.

FIG. 6 is a perspective view of a pneumatically-drivable mixing wheel suitable for use in a bioreactor.

FIG. 7 is a partial perspective view of the mixing wheel of FIG. 6, with select portions of the wheel omitted from the drawing to enable viewing of other portions of the drawing.

FIG. 8 is a schematic view of a bioreactor, including a fluid-containing vessel and a mixing wheel disposed within the vessel, having a volumetric capacity of 2 liters and identifying select physical dimensions of the bioreactor.

FIG. 9 is a schematic view of a bioreactor, including a fluid-containing vessel and a mixing wheel disposed within the vessel, having a volumetric capacity of 10 liters and identifying select physical dimensions of the bioreactor.

FIG. 10 is a schematic view of a bioreactor, including a fluid-containing vessel and a mixing wheel disposed within the vessel, having a volumetric capacity of 50 liters and identifying select physical dimensions of the bioreactor.

FIG. 11 is a schematic view of a bioreactor, including a fluid-containing vessel and a mixing wheel disposed within the vessel, having a volumetric capacity of 250 liters and identifying select physical dimensions of the bioreactor.

FIG. 12 is a schematic view of a bioreactor, including a fluid-containing vessel and a mixing wheel disposed within the vessel, having a volumetric capacity of 1000 liters and identifying select physical dimensions of the bioreactor.

FIG. 13 is a schematic view of a bioreactor, including a fluid-containing vessel and a mixing wheel disposed within the vessel, having a volumetric capacity of 2000 liters and identifying select physical dimensions of the bioreactor.

FIG. 14 is illustrates six bioreactors of progressively increasing capacity shown to relative scale.

FIG. 15 is a schematic representation of an alternative embodiment of the invention.

FIG. 16 is a schematic drawing of a cell culture system according to embodiments of the present invention.

FIG. 17 is a perspective view of an alternative embodiment of a mixing wheel in a flaps-closed configuration.

FIG. 18 is a perspective view of the alternative embodiment of the mixing wheel shown in FIG. 18 in a flaps-opened configuration.

FIG. 19 is a partial perspective view of an alternative embodiment showing both flaps-closed and flaps-opened configurations.

FIG. 20 is a first perspective view of a further alternative embodiment of a mixing wheel embodying aspects of the present invention.

FIG. 21 is a second perspective view of the mixing wheel of FIG. 19.

FIG. 22 is a perspective view of the mixing wheel of FIGS. 19 and 20 with an annular outer face of the mixing wheel omitted from the drawing.

### DETAILED DESCRIPTION

As used herein, the words “a” and “an” mean “one or more.” Furthermore, unless defined otherwise, all technical and scientific terms used herein have the same meaning as commonly understood by one of ordinary skill in the art to which the invention pertains. Although any methods and materials similar or equivalent to those described herein can be used in the practice of the present invention, the preferred materials and methods are described herein.

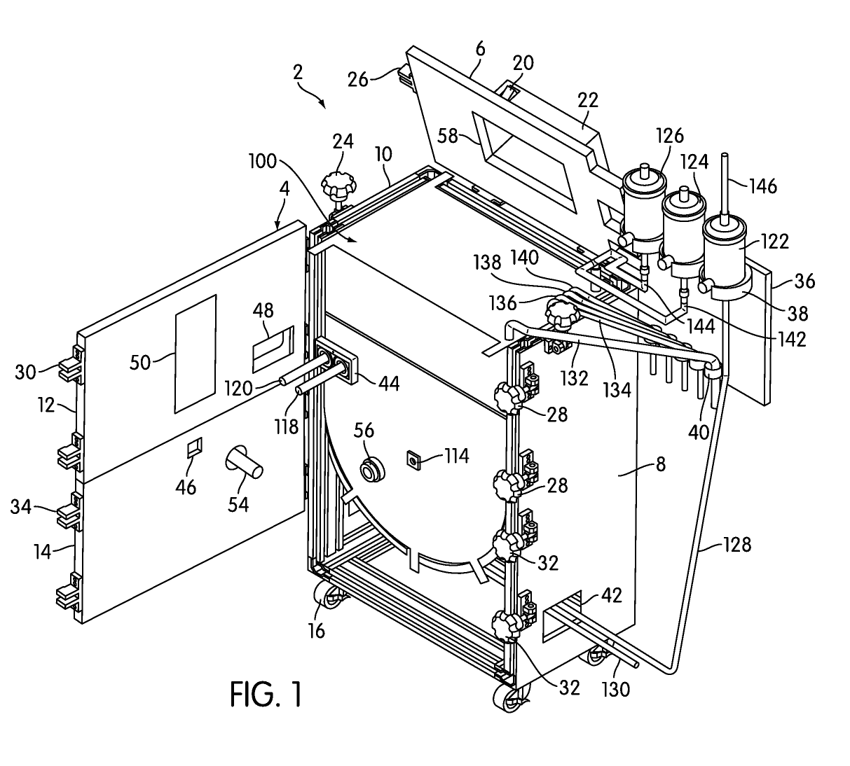

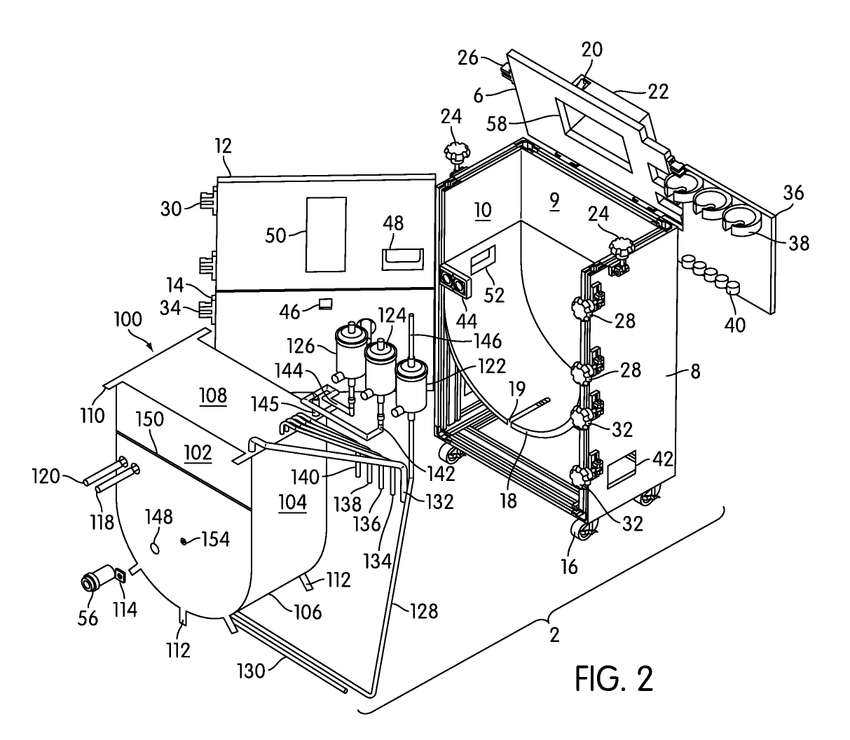

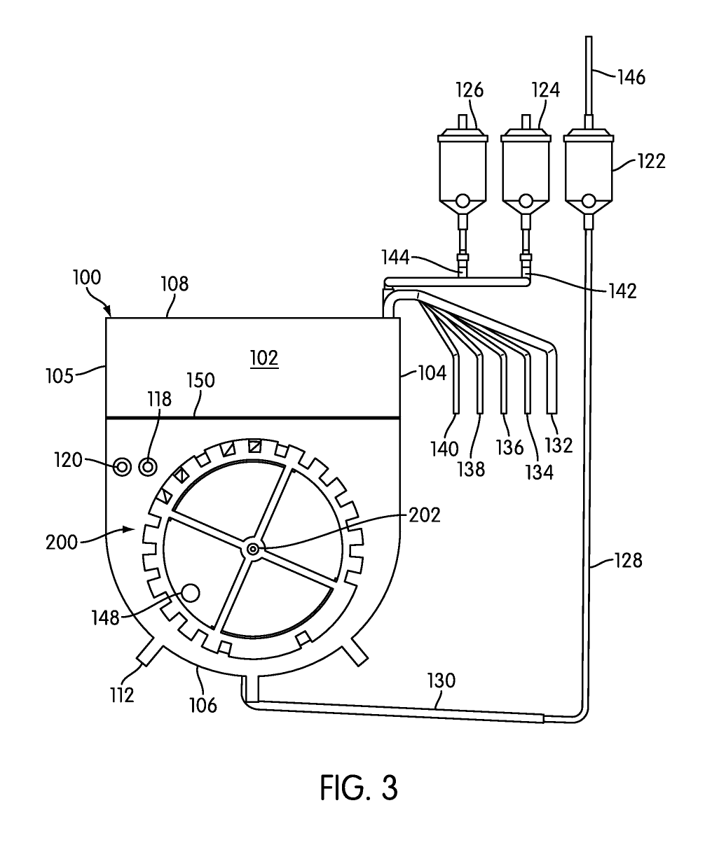

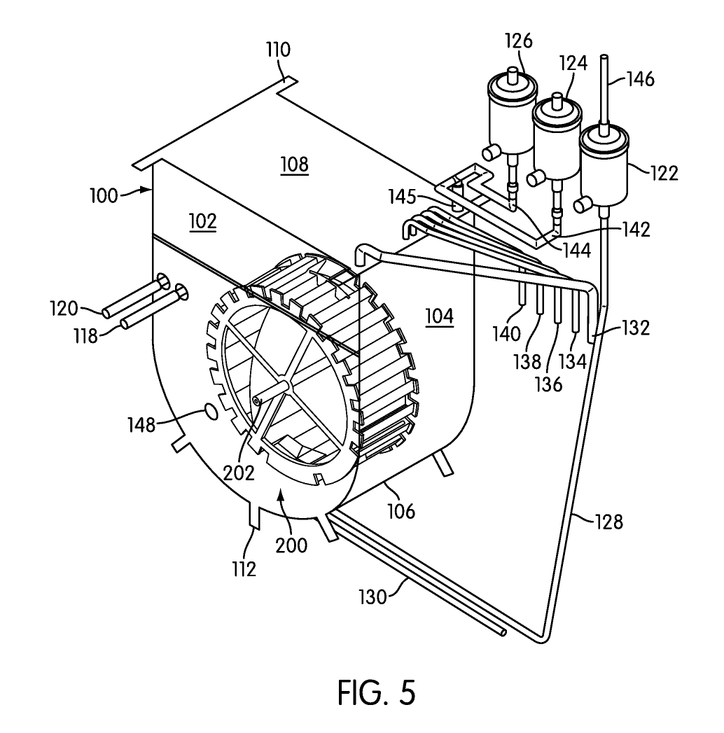

A bioreactor apparatus embodying aspects of the present invention is indicated by reference number 2 in FIGS. 1 and 2. The bioreactor apparatus comprises a housing 4 which contains and supports a reactor vessel 100 and includes various conduits and connections for attaching the apparatus 2 to a system for controlling and supplying the various gases and nutrients to support a reaction for cultivating cells (eukaryotic cells) or microbial organisms. As shown in FIGS. 3-5, a pneumatic mixing wheel 200 is disposed within the vessel 100 so as to be rotatable on shaft 202. Wheel 200 is described in more detail below.

Referring to FIGS. 1 and 2, housing 4 includes an upper lid 6 mounted with respect to the housing 2 on hinges or other similar components so as to permit the lid 6 to pivot between an open position, as shown in FIGS. 1 and 2, and a closed position. Housing 4 further includes a first side panel 8 (on the right hand side as shown in FIGS. 1 and 2) and a second side panel 10 (shown on the left hand side in FIG. 2). A rear panel 9 extends transversely between the parallel side panels 8 and 10. A front door includes an upper door 12 and lower door 14. Both the upper and lower doors 12, 14 are mounted with respect to the housing 4 by hinges or other similar components so as to enable the doors 12, 14 to pivot between open positions, as shown in FIGS. 1 and 2, and closed positions. A curved bottom wall 18 extends between the side panels 8 and 10. In the illustrated embodiment, curved bottom panel 18 is semicircular in shape. The curved bottom panel 18 conforms to a bottom panel of the vessel 100 (described below). A transverse slot 19 formed partially through the bottom panel 18 enables the vessel 100, with conduit connections extending from the bottom thereof, to be inserted into and removed from the housing 4. Housing 4 is preferably made from stainless steel.

The housing 4 further includes a light box 22 mounted to the top of the lid 6 and a switch 20 for controlling the light box 22. An opening 58 formed in the lid 6 allows the light from the light box 22 to illuminate the vessel 100 and its contents.

The lid 6 can be locked in a closed position by any suitable mechanism for holding the lid 6 in a closed position with respect to the housing 4. In the illustrated embodiment, the lid 6 is locked in a closed position with respect to the housing 4 by means of lock knobs 24 located on either side of the housing. Each lock knob 24 comprises a hinged threaded shaft with a hand wheel attached to each side panel 8 and 10 and which pivots with respect to a latch, or lock prong 26. The lock knobs 24 are pivoted down to permit the lid 6 to be closed. Then the lock knobs are pivoted upwardly to place the shaft of each knob within a corresponding prong 26. The hand wheel is turned to tighten the knob 24 down onto the lock prong 26, thereby securing the lid in the closed position.

The upper door 12 can be secured in a closed position by the same manner as the lid described above by similar hinged lock knobs 28 secured to the side panel 8 and engaged with the lock prongs 30 secured to the upper door 12. Lower door 14 can be secured in a closed position by lock knobs 32 attached to the side panel 8 and engaged with the lock prongs 34 attached to the lower door 14. As with lid 6, doors 12, 14 can be locked in a closed position by any suitable mechanism for holding the doors 12, 14 in a closed position with respect to the housing 4

Vessel 100 is preferably a disposable plastic bag that completely encloses the wheel 200. All valve and conduit attachments are sealed and filtered to keep the entire bag airtight and leak proof. Vessel 100 includes a front panel 102, an opposed parallel rear panel 103, a side panel 104, an opposed parallel side panel 105, a curved bottom panel 106, and a closed top panel 108. Preferably the curved bottom panel 18 of the housing 4 has a curvature conforming to the curvature of the curved bottom panel 106 of the vessel 100. Upper positioning tabs 110 are provided at the corners of the top panel 108, and lower positioning tabs 112 are provided at the bottom of the front panel 102 and the rear panel 103 along opposite edges of the curved bottom panel 106. The purpose of the positioning tabs 110, 112 will be described below. Vessel 100 may also include a liquid fill level line 150.

Suitable materials for constructing the vessel 100 include multi-layered or single-layered plastic films, including films made of polyethylene or Polyvinylidene Fluoride (PVDF) with thickness of 5 mil (0.127 mm) to 20 mil (0.508 mm) and most preferably 8 mil (0.2032 mm) to 12 mil (0.3048 mm). Various panels of the vessel are sealed to each other to form air-tight and water-tight seams by plastic film sealing techniques using heat, high radio frequency or other techniques. Then, the connectors, tubing, filters and closures are attached to the vessel to create the sterility barrier. The assembled vessel 100 may be sterilized by, for example, exposing the individual vessel to gamma irradiation, preferably between 25 to 50 K gray.

Alternatively, the vessel 100 may comprise a relatively rigid container that is, for example, formed by injection molding a suitable plastic, such as Polyethylene Terepthalate Glycol (PETG) and which may or may not be supported by an auxiliary structure, such as housing 4. Vessels comprising rigid containers may be preferable for relatively low-capacity bioreactors.

A shaft support button is secured to the exterior of the vessel 100 for supporting the shaft 202 of the mixing wheel 200. One shaft support button 114 is provided on each of the front panel 102 and the rear panel 103. Openings 154 are provided on the front panel 102 and the rear panel 103, and the support buttons 114 are secured to vessel 100 over the openings 154. The ends of the shaft 202 extend through openings 154 and into the support buttons 114.

A dissolved oxygen probe 118 and a pH probe 120 extend into the front panel 102 of the vessel 100. Gas to be added to the fluids within the vessel 100 is carried by conduit 146 into a gas inlet filter 122. From gas inlet filter 122, inlet gas line 128 carries gas to the bottom of the vessel 100. Vent gas is carried from the vessel 100 by the vent line 145, which branches into separate exhaust gas lines 142 and 144. Exhaust gas line 142 connects to the primary gas vent filter 124, and exhaust gas line 144 connects to the back up gas vent filter 126.

The gas filters should have sufficient gas flow capacity exceeding the inlet gas flow and be made of plastic filter material that is compatible with a gamma irradiation procedure, such as Polyvinylidene Fluoride (PVDF). Two vent filters are attached in a vessel in order to use one for normal operation and keep the second one as a back up in case of the failure of the first filter to avoid internal pressure build up. Suitable filters are available from the Pall Corporation, Millipore, or other manufacturers that provide products qualified for similar applications.

Conduit 130 is a culture broth harvest line for draining the culture broth from the vessel 100. Line 130 may also serve as a medium addition line for adding additional culture medium to the vessel 100.

Conduits 132, 134, 136, 138, and 140 communicate with the interior of vessel 100 through the top panel 108 of the vessel 100. Conduit 132 is a medium addition conduit, Conduit 134 is a base addition conduit, conduit 136 is a sampling line, conduit 138 is an inoculum/seed addition line, and conduit 140 is a line for nutrient feed medium addition.

In a preferred embodiment, the bioreactor apparatus 2 includes a mechanism for monitoring the speed of rotation of the pneumatic mixing wheel 200. In this regard, a magnetic sensor for a RPM meter may be attached to the front panel 102 of vessel 100 at location 148. The magnetic RPM sensor will detect the passage of a metal “flag” carried on the mixing wheel 200 to detect and monitor revolutions of the wheel.

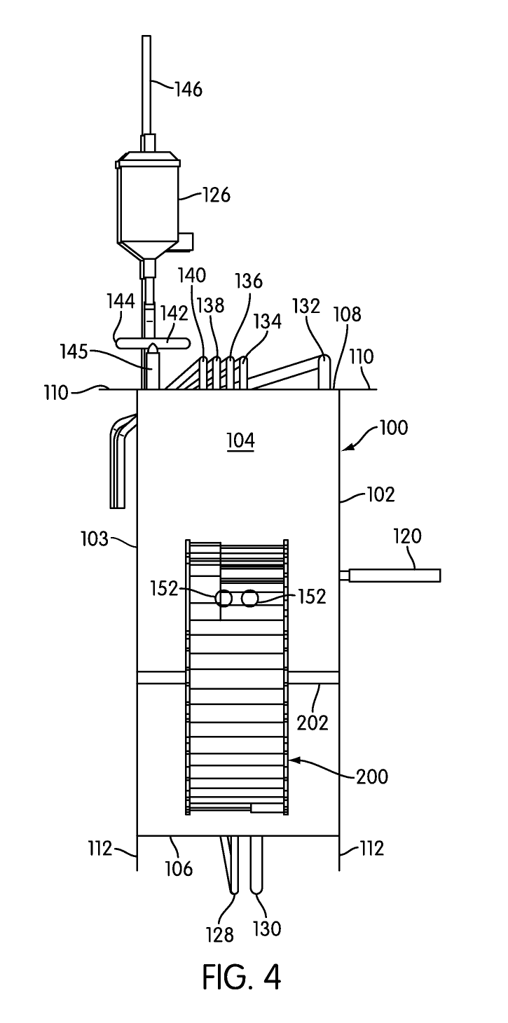

Vessel 100 also includes probe openings 152 (see FIG. 4) for disposable optic sensors to measure the pH or/and dissolved oxygen. The disposable optic sensors can be a back up to the pH probe 120 and the dissolved oxygen probe 118, or replace them if possible.

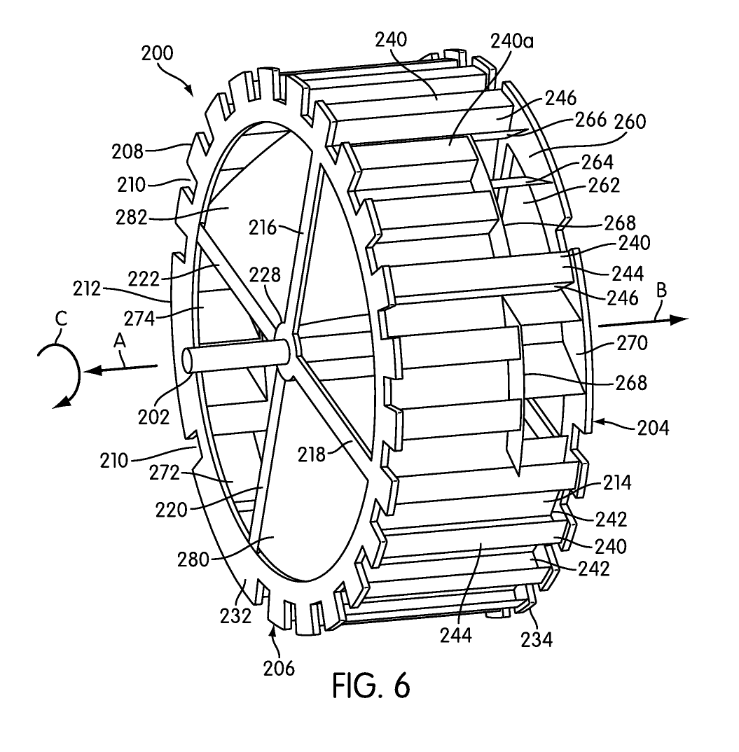

A pneumatic mixing wheel embodying aspects of the present invention is designated generally by reference number 200 in FIG. 6. The mixing wheel 200 is rotatably mounted on a shaft 202 and is defined generally by a wheel structure comprised of a first, radially oriented outer face 204 and a second, radially oriented outer face 206 spaced axially with respect to the first outer face 204. A cylindrical wall 214, oriented coaxially with the shaft 202, extends between the first outer face 204 and the second outer face 206.

First and second outer faces 204 and 206 have essentially identical structure. As shown in FIG. 6, second outer face 206 includes an outer annular portion 232 with a number of radial slots 210 about the periphery thereof so as to define small tabs 208 and large tabs 212 about the periphery of the outer face 206. Slots 210 facilitate lateral flow relative to the wheel 200. A number of spokes 216, 218, 220, 222 extend radially inwardly from the annular portion 232 of the outer face 206 connecting the annular portion 232 with a shaft ring 228 within which the shaft 202 is rotatably mounted. In the illustrated embodiment, the second outer face 206 includes four spokes 216, 218, 220, 222, spaced equally by 90 degrees. Other numbers of spokes may be implemented.

The pneumatic mixing wheel 200 further includes a plurality of gas entrapment elements positioned about the outer periphery of the cylindrical wall 214 and configured to capture at least a portion of the gas introduced into the vessel 100 by the gas inlet line 128 and cause the mixing wheel 200 to rotate due to the buoyancy of the entrapped by the gas-entrapment cups 240 formed about the outer periphery of the wheel, projecting outwardly from the cylindrical wall 214. Gas-entrapment elements may comprise vanes or paddles configured to capture rising gas bubbles In the illustrated embodiment, the gas entrapment elements comprise gas-entrapment cups 240. Each cup 240 comprises a partial enclosure formed by an outer wall 244, a back wall 246, a portion of the cylindrical wall 214, and two tabs 208 or 212 of the first and second outer faces, 204, 206 disposed at opposite ends of the cup 240. A circumferentially facing opening 242 is defined at an open end of the cup 240. The openings 242 of all of the cups 240 are oriented in the same direction in order to entrap rising gas bubbles released from a source (e.g., inlet gas line 128) entering the vessel 100 beneath the mixing wheel 200. The buoyancy of the trapped rising gas will cause each cup 240 along one side of the wheel 200 (the side of the wheel furthest from the viewer in FIG. 6) to rise, thereby causing the entire wheel to rotate around its axis in the direction indicated by clockwise arrow C. As the wheel continues to turn, the cup openings 242, facing downwardly in order to trap gas along one side of the wheel, become oriented upwardly on the opposite side of the wheel (the side closest to the viewer in FIG. 6), thereby releasing the trapped gas.

The gas cups 240 shown in FIG. 6 have a rectangular, box-like shape, but cups having other shapes, such as hemispherical, semi-oval, or triangular, may be implemented.

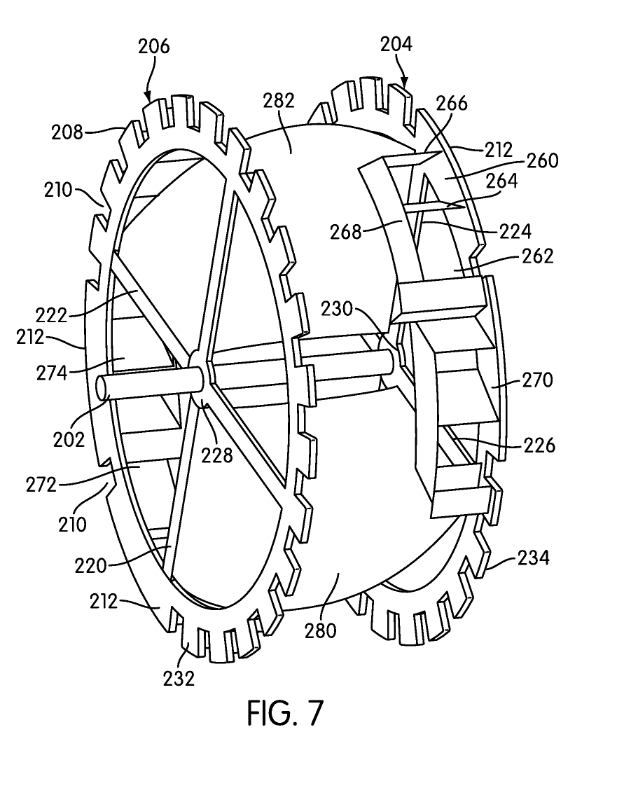

As shown in FIG. 7, in which the cylindrical wall 214 and gas entrapment cups 240 located between the first outer face 204 and the second outer face 206 are omitted, the first outer face 204 also includes spokes (spokes 224 and 226 being visible and numbered) which connect annular portion 234 of the first outer face 204 with a shaft ring 230 within which the shaft 202 is rotatably inserted.

The mixing wheel 200 further includes radial deflectors 260, 270, 272, and 274, and axial deflector vanes 280 and 282.

Radial deflector 260 includes an opening 262 formed in the cylindrical wall 214 and radial inflow elements disposed within the opening 262 and extending between a large tab 212 of the first outer face 204 and a peripherally extending, radially oriented side wall 268. The opening 262 extends between the first outer face 204 and the side wall 268. The radial inflow elements, 264, 266 comprise vanes which are angled in a direction of rotation of the wheel as defined by the orientations of the gas entrapment cups 240. As the wheel 200 rotates in the direction indicated by arrow C under the buoyant force of trapped gas bubbles within the gas cups 240, a portion of the fluid around the outer periphery of the wheel 200 is deflected by the angled vanes 264 and 266 through the opening 262 and caused to flow radially inwardly, into the interior portion of the cylindrical wall 214, in a direction generally toward the shaft 202.

In one embodiment, radial deflector 260 and opening 262 has an axial width of approximately one-third the overall axial width of the cylindrical wall 214 of the wheel 200. Accordingly, the gas entrapment cups, indicated by reference number 240a, adjacent the radial deflector 260 are axially shorter than cups 240 located elsewhere on the wheel 200 and have an axial length of approximately two-thirds the length of the gas cups 240.

In the illustrated embodiment, there are four radial deflectors. Radial deflectors 270, 272, and 274 have a structure that is identical to that of radial deflector 260. Also, two radial deflectors 260, 270 are preferably located at one axial edge of the wheel 200 (the right-hand side of the wheel shown in FIG. 6), and two radial deflectors 272, 274 are located on the opposite axial side of the wheel 200 (the left-hand side of the wheel shown in FIG. 6).

The axial outflow elements comprise curved deflector vanes 280 and 282 disposed within the interior of cylindrical wall 214. Each deflector vane 280, 282 extends from a spoke of the first outer face 204 to a spoke on the second outer face 206 that is displaced 90° from the first spoke. More specifically, as shown in FIG. 7, deflector vane 280 extends from spoke 226 of the first outer face 204 (oriented horizontally in the figure) to the spoke 220 of the second outer face 206 (oriented vertically in the figure). Similarly, deflector vane 282 extends from spoke 224 of the first outer face 204 to spoke 222 of the second outer face 206.

The deflector vanes 280, 282 are disposed with respect to the radial deflectors 260, 270, 272, 274 to redirect in a generally axial direction flow that has been redirected by the radial deflectors into the interior of the cylindrical wall 214. More specifically, fluid flow directed radially inwardly by the radial deflector 260 will impinge upon a first side of the deflector vane 282 and be redirected in a generally axial direction corresponding to arrow A in FIG. 6. Similarly, fluid flow directed radially inwardly by the radial deflector 274 will impinge upon an opposite side of the deflector vane 282 and be redirected in a generally axial direction corresponding to arrow B in FIG. 6. Similarly, fluid flows directed radially inwardly by radial deflectors 270 and 272 will impinge upon opposite sides of the deflector vane 280 and be directed in axial directions A and B.

Wheel 200 and shaft 202 can be made from any suitable non-reactive material, including, for example, polycarbonate, Polyethylene Terepthalate Glycol (PETG), PVDF, or any other plastic material suitable for cell culture processes.

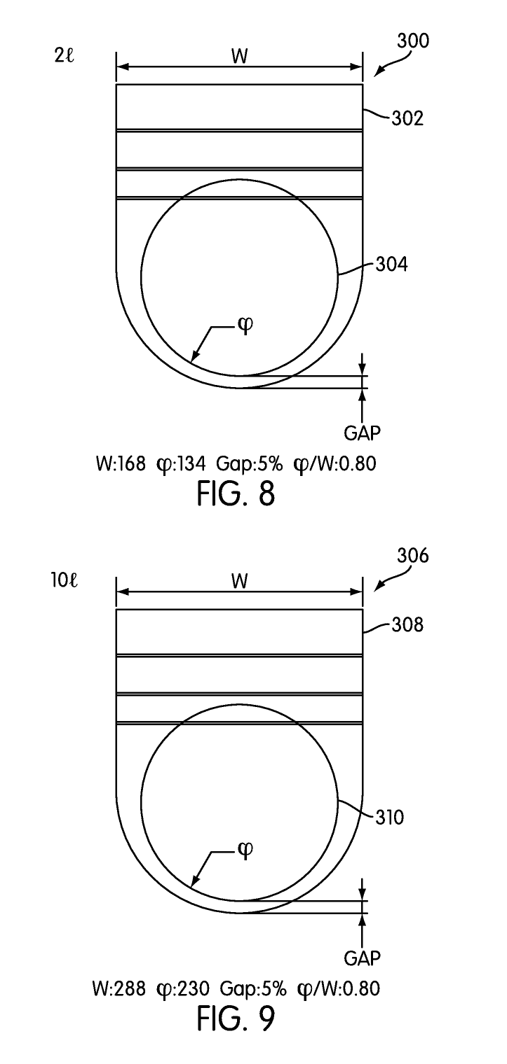

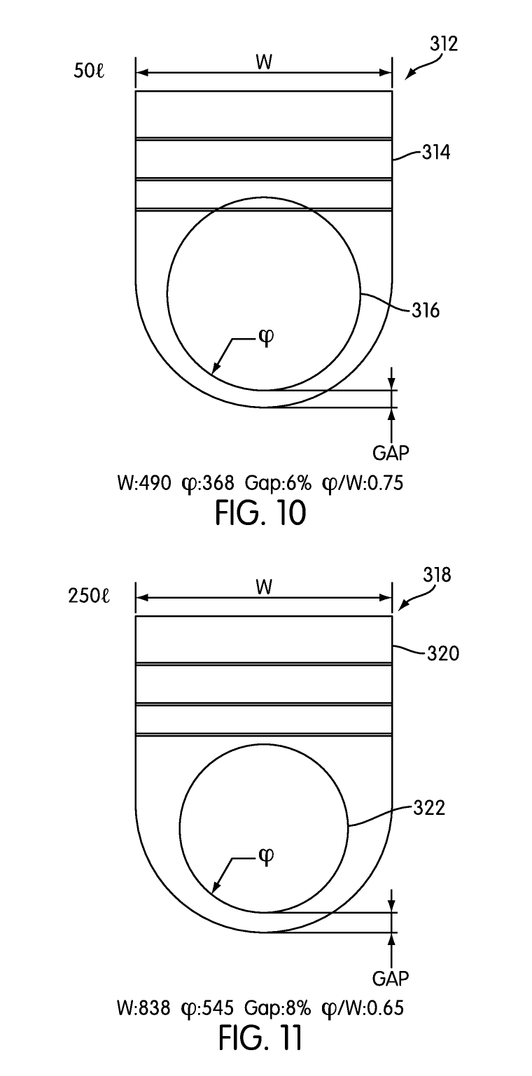

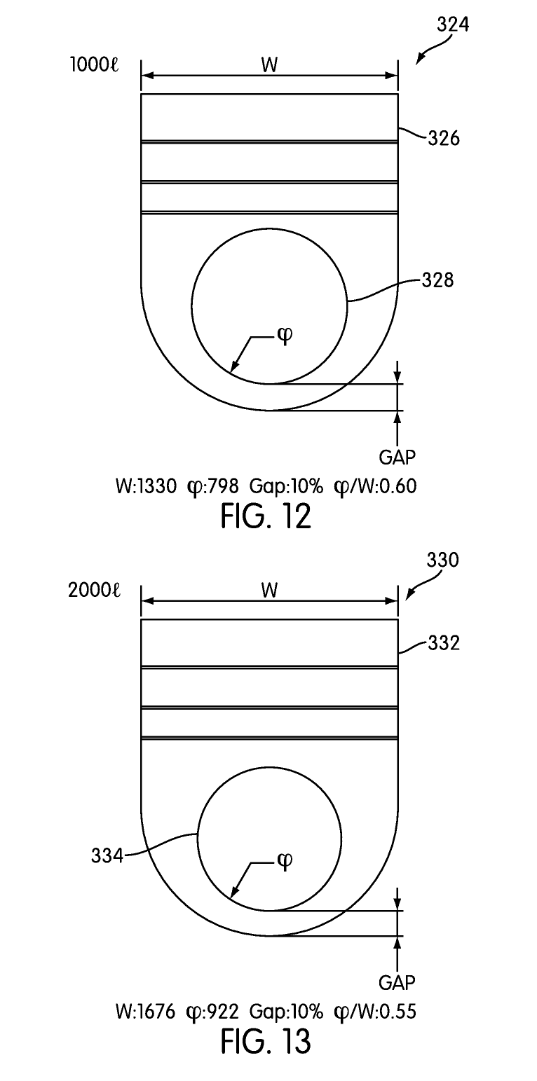

Another aspect of the invention is illustrated in FIGS. 8-14. FIGS. 8-14 show schematically bioreactors of increasing volumetric capacities with many features of the bioreactors, such as the housing and conduits, removed for clarity. Bioreactor 300 shown in FIG. 8 has a capacity of 2 liters, bioreactor 306 shown in FIG. 9 has a capacity of 10 liters, bioreactor 312 shown in FIG. 10 has a capacity of 50 liters, bioreactor 318 shown in FIG. 11 has a capacity of 250 liters, bioreactors 324 shown in FIG. 12 has a capacity of 1000 liters, and bioreactor 330 shown in FIG. 13 has a capacity of 2000 liters.

Referring to FIG. 8, the bioreactor 300 includes a vessel 302 having a capacity of 2 liters and a pneumatic mixing wheel 304. The vessel 302 has a transverse dimension W of 168 mm, and the mixing wheel 304 has a transverse dimension, or diameter, Φ of 134 mm. The ratio of Φ/W is 0.80, and the gap between the lower most portion of the mixing wheel 304 and the bottom of the vessel 302 is 8.4 mm or 5% of the transverse dimension W=168 mm.

Referring to FIG. 9, the bioreactor 306 includes a vessel 308 having a capacity of 10 liters and a pneumatic mixing wheel 310. The vessel 308 has a transverse dimension W of 288 mm, and the mixing wheel 310 has a transverse dimension, or diameter, Φ of 230 mm. The ratio of Φ/W is 0.80, and the gap between the lower most portion of the mixing wheel 310 and the bottom of the vessel 308 is 14.4 mm or 5% of the transverse dimension W=288 mm.

Referring to FIG. 10, the bioreactor 312 includes a vessel 314 having a capacity of 50 liters and a pneumatic mixing wheel 316. The vessel 314 has a transverse dimension W of 490 mm, and the mixing wheel 316 has a transverse dimension, or diameter, Φ of 368 mm. The ratio of Φ/W is 0.75, and the gap between the lower most portion of the mixing wheel 316 and the bottom of the vessel 314 is 29.4 mm or 6% of the transverse dimension W=490 mm.

Referring to FIG. 11, the bioreactor 318 includes a vessel 320 having a capacity of 250 liters and a pneumatic mixing wheel 322. The vessel 314 has a transverse dimension W of 838 mm, and the mixing wheel 322 has a transverse dimension, or diameter, Φ of 545 mm. The ratio of Φ/W is 0.65, and the gap between the lower most portion of the mixing wheel 322 and the bottom of the vessel 320 is 67 mm or 8% of the transverse dimension W=838 mm.

Referring to FIG. 12, the bioreactor 324 includes a vessel 326 having a capacity of 1000 liters and a pneumatic mixing wheel 328. The vessel 326 has a transverse dimension W of 1330 mm, and the mixing wheel 328 has a transverse dimension, or diameter, Φ of 798 mm. The ratio of Φ/W is 0.60, and the gap between the lower most portion of the mixing wheel 328 and the bottom of the vessel 326 is 133 mm or 10% of the transverse dimension W=1330 mm.

Referring to FIG. 13, the bioreactor 330 includes a vessel 332 having a capacity of 2000 liters and a pneumatic mixing wheel 334. The vessel 332 has a transverse dimension W of 1676 mm, and the mixing wheel 334 has a transverse dimension, or diameter, Φ of 922 mm. The ratio of Φ/W is 0.55, and the gap between the lower most portion of the mixing wheel 334 and the bottom of the vessel 332 is 167.6 mm or 10% of the transverse dimension W=1676 mm.

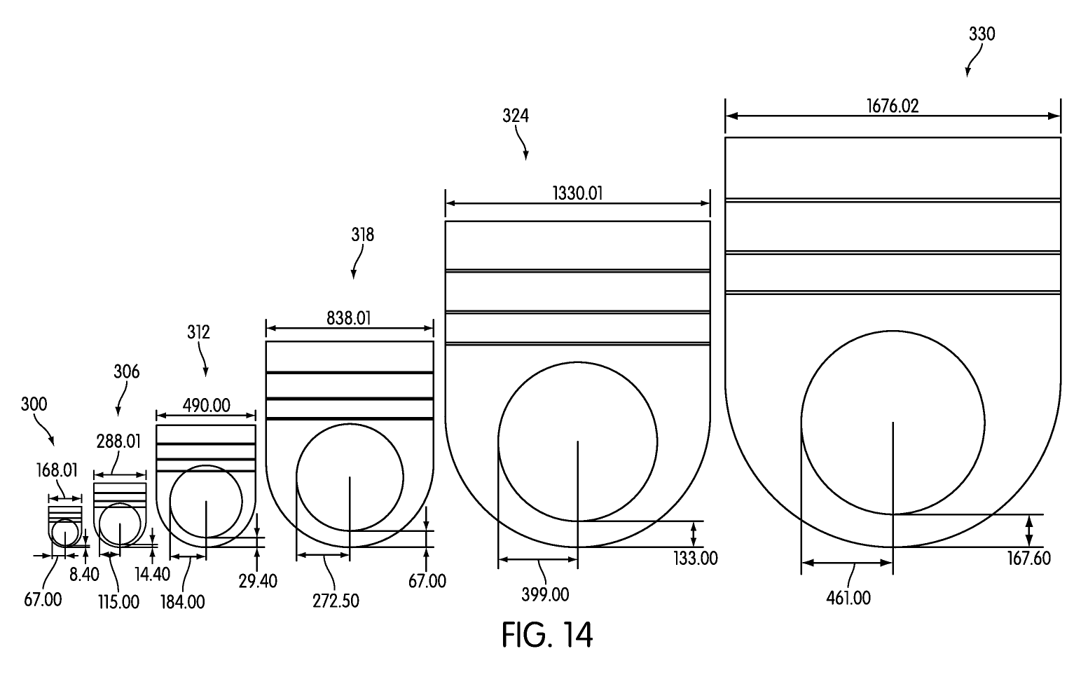

FIG. 14 shows the bioreactors 300, 306, 312, 318, 324, and 330 of increasing capacity side-by-side, generally to relative scale so as to show the differences in sizes between the reactors. As can be appreciated from the description above and from FIG. 14, in general, as the capacity of the bioreactor increases, the transverse dimension (i.e., diameter) of the mixing wheel relative to the transverse dimension (width) of the vessel decreases so that the ratio Φ/W decreases as the capacity increases. Similarly, in general, the gap between the lower most portion of the mixing wheel and the bottom of the vessel increases as the capacity of the bioreactor increases.

The inventors have discovered that as the capacity of the bioreactor vessel increases, it is not necessary to incorporate a corresponding increase in the diameter of the mixing wheel. As the capacity of the bioreactor increases, the volume of gas that must be added to the vessel to support cell culture growth increases. The increased gas supply generates a faster rotation of the pneumatic mixing wheel. Also, as the diameter of the mixing wheel increases, the radial speed of the outer periphery of the wheel increases for a given rate of rotation. The cumulative result of these effects is that as the volume capacity of the bioreactor increases, adequate mixing can be achieved by a relatively smaller pneumatic mixing wheel to the size of vessel.

And, moreover, it is not necessary that the wheel be positioned as close to the bottom of the vessel to ensure thorough mixing (and avoid dead zones) as the volume of the bioreactor increases.

FIG. 14 demonstrates the scalability of the bioreactor. As volumetric capacity of the bioreactor increases, neither the wheel radius nor the gap distance increases linearly with the width of the vessel. For larger volumes, the wheel is relatively smaller compared to the vessel size. For a set amount of gas flow, the rotational force of a larger wheel will be greater (due to the larger radius and increased wheel speed at the circumference of the wheel), so greater particle suspension power is achieved at the bottom of the vessel. Therefore, the gap between the curved bottom of the vessel and the wheel can be greater without sacrificing effective mixing in that area (one of the most important aspects of mixing due to gravity causing settling at the bottom of the bag).

While keeping the gas flow rate (volume per volume per minute) constant leads to faster mixing at higher volumes, this phenomenon can also be applied in reverse: if it is desired to maintain the same mixing power across all volumes, then larger wheel sizes require less gas flow, or smaller size of wheel can be used for the larger vessel using the same gas flow rate. Advantage of having smaller size wheel is to make the packaging, delivery and handling of disposable bioreactors easier.

Another key feature of these bioreactors is their ability to be linked in sequence, connecting the output of one bioreactor apparatus to the input of the next larger bioreactor apparatus. This sequential size of bioreactors allows use of the disposable bioreactors for the entire seed train as well as the production stage.

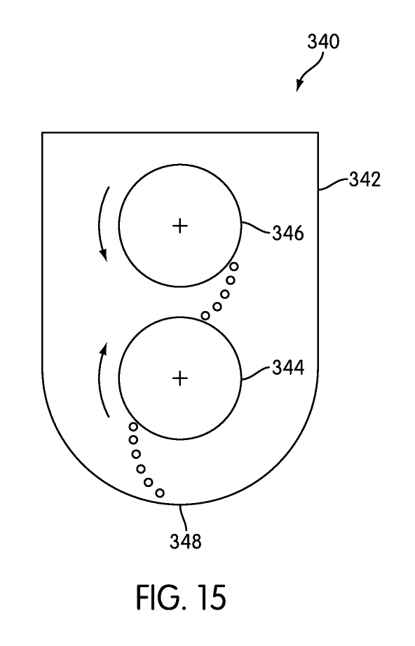

An alternative arrangement of the bioreactor is indicated by reference number 340 in FIG. 15. Bioreactor 340 includes a vessel 342 and two pneumatic mixing wheels 344, 346. Mixing wheels 344, 346 are arranged generally one above the other, and are positioned above a location 348 at which air or other gas is introduced into the vessel 342. Gas bubbles impinge on the first (lowest) wheel 344 and the buoyancy of the bubbles causes rotation of the wheel (e.g., clockwise). As wheel 344 continues to rotate, gas bubbles are released from the downwardly moving side of the wheel. Those released bubbles impinge upon the second wheel 346, and cause rotation of the second wheel 346 in a direction (e.g., counter-clockwise) opposite the direction of rotation of the first wheel 344.

The bubbles are trapped on one side of the wheel and are released on the opposite side of the wheel. The second wheel 346 must be configured to trap the bubbles released by the first wheel 344. Thus, the bubble-trapping side of second wheel 346 is positioned generally above the bubble-releasing side of first wheel 344. In the illustrated embodiment, this means that the openings of the gas cups (see, e.g., gas cups 242 in FIG. 6) of the first wheel 344 and the openings of the gas cups of the second wheel 346 must face in opposite directions.

It can be appreciated that more than two pneumatic mixing wheels can be stacked one above the other in the manner shown in FIG. 15, with each wheel (except the top wheel) releasing bubbles to the wheel above it, and each wheel (except the bottom wheel) trapping bubbles released from the wheel below it.

Another alternative arrangement may comprise two or more mixing wheels arranged end-to-end so as to be rotatable about a common axis.

Referring primarily to FIGS. 1 and 2, installation of the vessel 100 into the housing 4 will now be described. The empty vessel 100 is placed within the open housing 4 with the curved bottom panel 106 of the vessel 100 supported upon the curved bottom wall 18 of the housing 4. Connection tubes extending from the bottom of the vessel 100 for connecting the inlet gas line 128 and the harvest line 130 extend through the slot 19 formed in the bottom wall 18.

The inlet gas line 128 and the harvest line 130 are connected to the bottom of the vessel 100 and extend through an opening 42 formed in the side panel 8 of the housing 4. The dissolved oxygen probe 118 and the pH probe 120 are connected to the vessel 100 using a connector 44 which projects through or is secured within an opening 48 formed in the upper door 12. A probe valve 54, which allows alignment of an RPM meter, is inserted into a receptacle 56 attached to the vessel 100.

Conduits 132, 134, 136, 138, and 140 are attached by suitable connectors to the top panel 108 of the vessel 100 and are supported by conduit guides 40 mounted on a support panel 36 extending laterally from the housing 4. Filter elements 122, 124, and 126 are carried on filter supports 38 also attached to the support panel 36. The exhaust conduit 145 is also connected by a suitable connector to the top panel 108. At this point, the doors 12 and 14 and the lid 6 can be closed and locked. A recess 46 formed on the inside of the lower door 14 receives and holds the shaft support button 114. A similar recess is formed on the rear panel 9 of the housing 4 for holding the shaft support button 114 attached to rear panel 103 of vessel 100. The shaft 202 of the wheel 200 extends through the openings 154 formed in the front panel 102 and rear panel 103 of the vessel and are supported within the shaft support buttons 114 which in turn are carried within the recesses 46 of the housing 4. Accordingly, wheel 200 is supported for rotation about a substantially horizontal axis corresponding with the shaft 202. Furthermore, in an embodiment of the bioreactor, the axis of rotation defined by shaft 202 is parallel to an axis defining the center of curvature of curved panel 106 of vessel 100.

The upper positioning tabs 110 and the lower positioning tabs 112 help flatten the bag surfaces. When the lid 6 and the doors 12, 14 are closed, they pinch the tabs 110, 112, thereby securing the bag in place within the housing 4. With the vessel 100 secured within the housing 4, the vessel can be filled, preferably up to the fill line 150. A glass window 50 provided in the upper door 12 permits an operator to look into the vessel after the doors 12, 14 and lid 6 are closed.

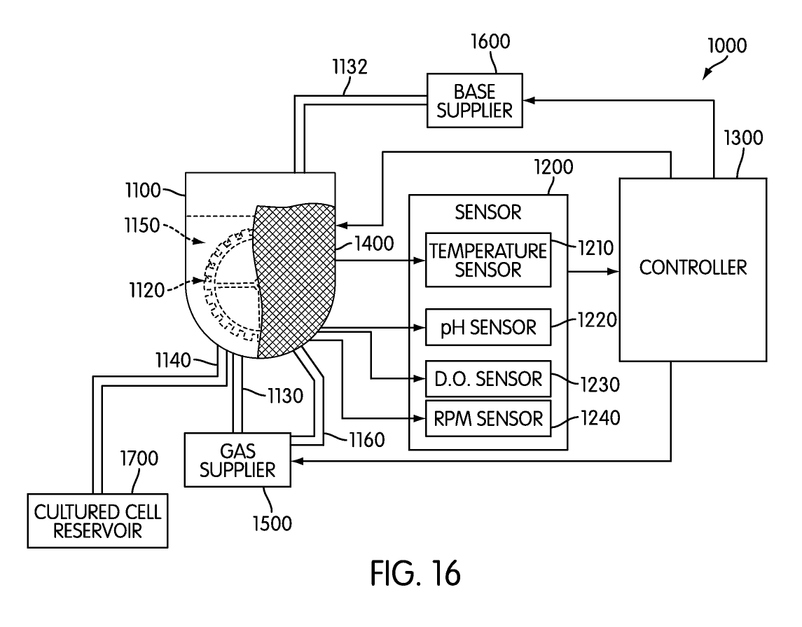

FIG. 16 is a schematic drawing of a bioreactor system 1000 according to aspects of the present invention.

Referring to FIG. 16, the bioreactor system 1000 comprises a bioreactor apparatus 1100 described above with reference to FIGS. 1-7, sensor module 1200, controller 1300, heating element 1400 (which may comprise heating elements within the housing supporting a disposable vessel), gas supply module 1500, and base supply module 1600.

Sensor module 1200 may include a number of sensors for sensing various parameters associated with the cell culture procedure. For example, sensor module 1200 may include a temperature sensor 1210 for sensing temperature of culture medium 1150 contained in a bioreactor apparatus 1100. Module 1200 may further include a pH sensor 1220 for sensing pH of the medium 1150 and a dissolved oxygen (“D.O.”) sensor 1230 for sensing the concentration of dissolved oxygen of the medium 1150. D.O. sensor 1230 and pH sensor 1220 may comprise dissolved oxygen probe 118 and pH probe 120, respectively. Sensor module 1200 may further include an RPM sensor 1240 (incorporated into probe valve 54) for sensing rotation (e.g., RPM) of the wheel 1120. Sensors included in sensor module 1200 and the sensing process may be based on conventional sensing methods and devices.

Controller 1300 receives signals from the sensors of sensor module 1200, and generates and transmits a plurality of control signals. Heating elements 1400 may be partially or wholly attached to the outer side of a bioreactor apparatus 1100 to supply heat by a temperature control signal included in the plurality of control signals to maintain constant temperature of culture medium 1150.

Heating element 1400 may comprise a conventional water jacket or other conventional heating elements, such as resistive foils. In an embodiment of the present invention, bioreactor apparatus 1100 may comprise no heating elements.

Gas supply module 1500 supplies oxygen, nitrogen, carbon dioxide, air or a mixture thereof directly supplied to culture medium 1150 through a gas inlet 1130 or/and a separate gas sparging line 1160. Gas supply module 1500—which may comprise filter 122, inlet conduit 146, and gas supply conduit 128—is controlled by controller 1300 and a dissolved oxygen concentration signal from D.O. sensor 1230, a pH signal from pH sensor 1220, and an RPM signal from RPM sensor 1240, while controlling the concentration of the gas. As the speed of the pneumatic mixing wheel is governed by the rate of gas flow into the apparatus 1100, controller 1300, based on signals from the RPM meter, controls gas flow rate and gas volume to optimize wheel rotation and mixing. Also, the controller 1300 monitors gas concentrations, e.g., from the dissolved oxygen probe 118 and the pH probe 120, and, based thereon, controls the input gas mixture—by mixing oxygen, carbon dioxide, and an inert gas, to optimize cell growth. Accordingly, the controller 1300, together with the gas supply module 1500, monitors gas concentrations and wheel rotation and optimizes the gas flow rate and composition to optimize mixing and cell growth.

A base supply module 1600 supplies base solution to the culture medium 1150 through a nutrient inlet 1132 (which may comprise conduits 138 and 140) and is controlled by controller 1300 and a pH signal from pH sensor 1220.

Further, in another embodiment of the present invention, a bioreactor system 1000 further comprises a cultured cell reservoir 1700. In this case, bioreactor apparatus 1100 further comprises a culture medium outlet 1140 (which may comprise harvest line 130) for discharging culture medium 1150. A cultured cell reservoir 1700 discharges culture medium 1150 including cells from bioreactor apparatus 1100 through the culture medium outlet 1140 and stores it.

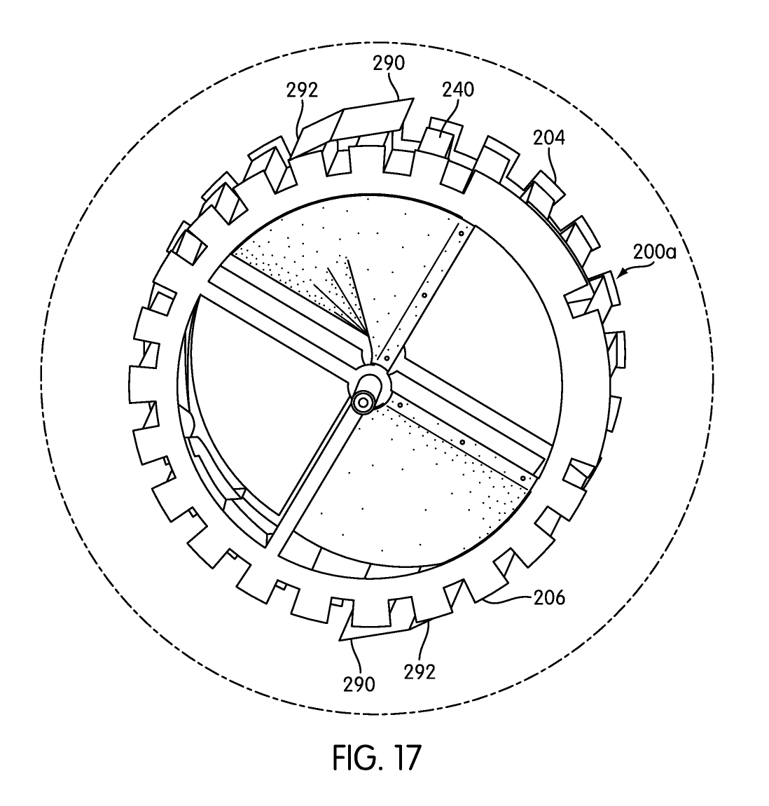

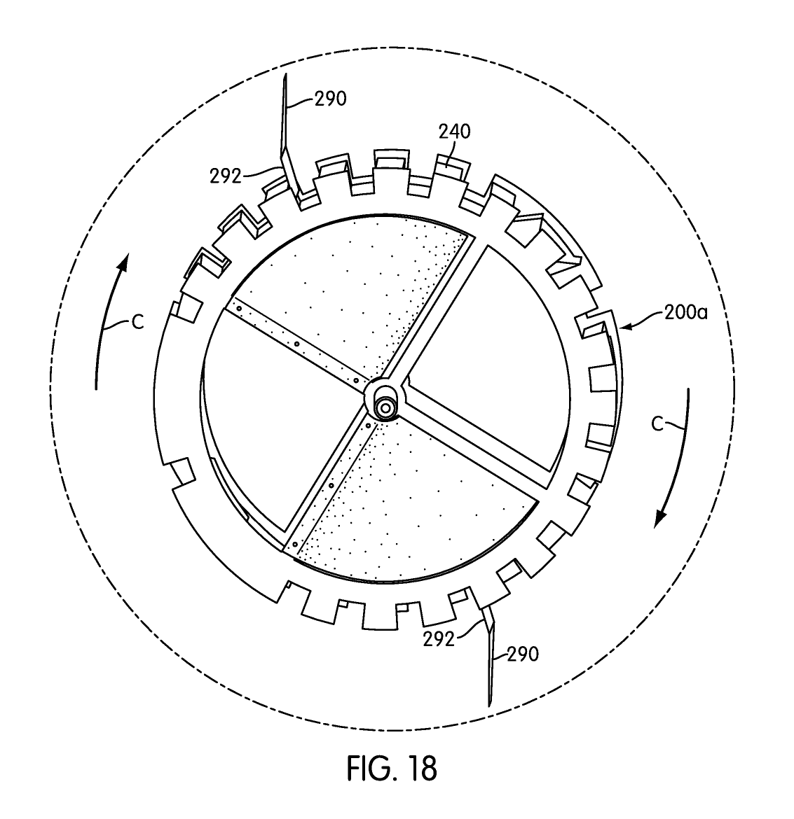

An alternative configuration of a pneumatic mixing wheel is indicated by reference number 200a in FIGS. 17 and 18. Components of mixing wheel 200a of FIGS. 17 and 18 that are identical with components of mixing wheel 200 of FIGS. 6 and 7 have like reference numbers.

Although it is necessary for the diameter of the mixing wheel to increase with the vessel size to ensure sufficient, homogeneous fluid mixing to avoid cell sedimentation at the bottom and dead zones at the top corners of the vessel, it is also desirable to minimize the overall bag dimensions for purposes of gamma sterilization, shipping, and storage, especially for larger-sized bags that are 250 L working volume and greater. During sterilization, shipping and storage, the flexible bag vessel is collapsed around the mixing wheel disposed within the interior of the bag. Therefore, the compactness that can be achieved when collapsing the vessel is limited by the size of the mixing wheel.

Thus, in the embodiment of FIGS. 17 and 18, the mixing wheel 200a, especially for use in large volume bioreactor vessels, is equipped with one or more “flaps” 290 that are hinged at end 292 thereof either on the gas cups 240 or on the edge of the first and second outer faces, 204, 206. Although only two diametrically-opposed flaps 290 are shown in FIGS. 17 and 18, mixing wheel 200a may include a plurality of flaps 290 evenly spaced about the perimeter of the wheel for symmetric weight distribution. The flaps 290 lie flat along the outer perimeter of the wheel 200a in a flaps-closed configuration during sterilization, shipping, and storage to minimize the size of the wheel (FIG. 17) but unfold and extend out radially in a flaps-open configuration during cell culture operation as gas is introduced into the vessel and the wheel 200a begins to rotate in direction “C” (FIG. 18). The hinge of the flaps 290 would be designed to engage and lock into place (for example, by a detent or similar mechanism) once the flaps 290 are extended out to ensure the flaps remain in the open configuration. Such a locking mechanism should be strong enough to hold the flaps 290 in the extended position against the drag force by the liquid.

The shape of the flaps could also be customized to generate different flow patterns, depending on the desired application. The flaps may be substantially flat, as shown, for maximum tangential fluid flow, or curved and angled to provide additional radial and axial flow. One or more openings can be formed through the flap 290 to reduce the drag induced by the flap and/or to generate turbulence to enhance the mixing effect of the flap 290.

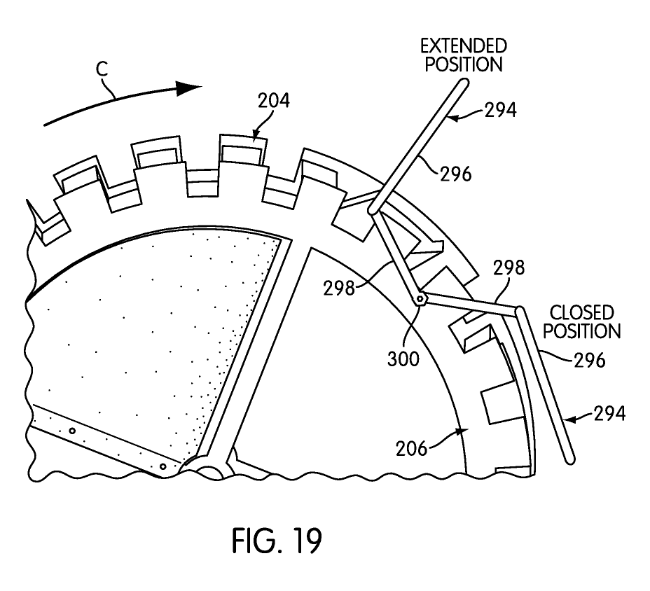

An alternative configuration of an extendible flap is shown in FIG. 19. In FIG. 19, flap 294 is shown in a closed position and an opened position. Flap 294 includes a panel portion 296 and an extended frame comprising arms 298 (only one arm is shown in FIG. 19) which extend from an end of the panel portion 296 and are connected at a pivot point 300 to first and second outer faces, 204, 206 of the wheel. In the closed position, the flap 294 lies flat against the perimeter of the wheel. When the wheel rotates in direction “C”, the drag of the fluid against the panel portion 296 rotates the flap 294 about pivot point 300 to the flaps opened position, and the drag holds the flap 294 in the flaps opened position as long as the wheel is rotating.

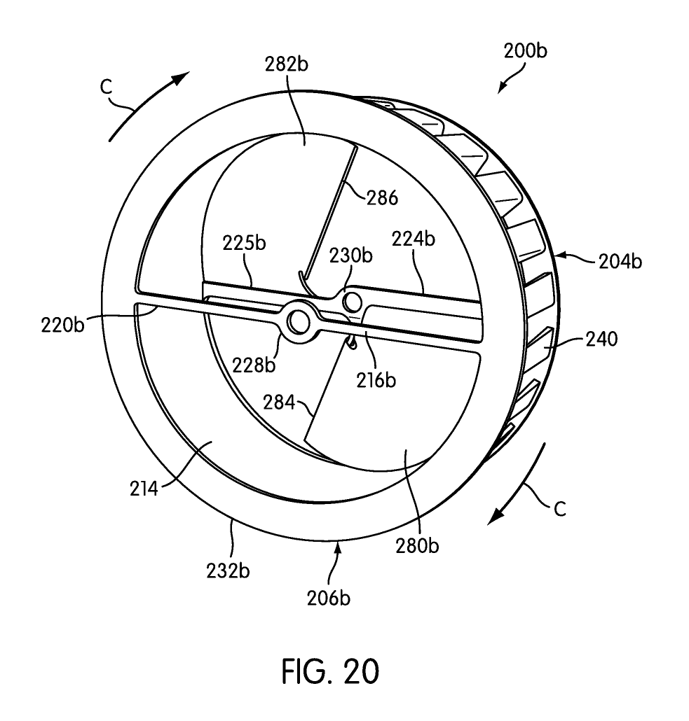

A further alternative embodiment of a pneumatically-driven mixing wheel embodying aspects of the present invention is indicated by reference number 200b in FIGS. 20-22. Components of mixing wheel 200b of FIGS. 20-22 that are identical with components of mixing wheel 200 shown in FIGS. 6 and 7 have like reference numbers.

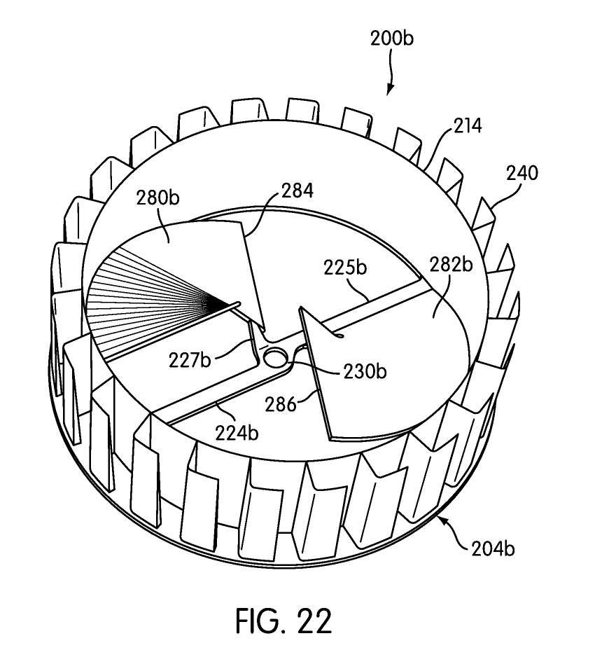

Mixing wheel 200b includes a cylindrical wall 214, a first outer face 204b and a second outer face 206b. Second outer face 206b is omitted from FIG. 22 to facilitate viewing of various structures of the wheel 200b. Second outer face 206b comprises an annular portion 232b and two diametrically opposed spokes 216b, 220b which connect to a shaft ring 228b. First outer face 204b also includes spokes 224b, 225b which connect to a shaft ring 230b and may be of a different configuration than the spokes and shaft ring of second outer face 206b. Mixing wheel 200b further includes a plurality of gas entrapment elements, such as gas-entrapment cups 240, disposed about the periphery of the cylindrical wall 214.

Mixing wheel 200b does not include radial inflow elements, such as radial deflectors 260, 270, 272, 274 shown in FIGS. 6 and 7. Instead, mixing wheel 200b includes deflector vanes 280b and 282b disposed in the interior portion of the cylindrical wall 214 and having leading edges 284, 286, respectively, that extend axially beyond the first and second outer faces 204b, 206b, respectively. Deflector vane 280b extends from spoke 216b of second outer face 206b and curves over an arch of greater than 90 degrees to its leading edge 284, which extends axially beyond the first outer face 204b. First outer face 204b includes a deflector support arm 227b extending from the shaft ring 230b to a portion of the deflector vane 280b. Deflector vane 282b extends from spoke 225b of first outer face 204b over an angle of more than 90 degrees to its leading edge 286 which extends axially beyond the second outer face 206b.

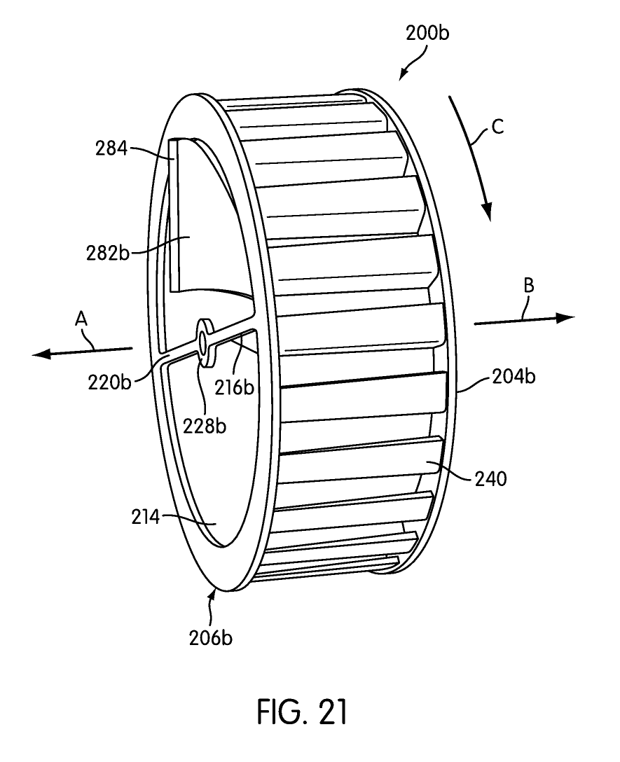

As the mixing wheel 200b rotates in the direction indicated by arrow C due to the buoyancy of gas trapped in the gas entrapment cups 240, as described above, the leading edges 286 and 284 of the deflector vanes 280b and 282b direct fluid that is exterior to the cylindrical wall 214 into the interior of the cylindrical wall 214 and in a direction generally axial with respect to the cylindrical wall 214. Accordingly, the mixing wheel 200b generates axial flow away from the wheel 200b in the directions indicated by arrows A and B in FIG. 21, and that axial flow will impinge upon the front and rear walls 102, 104 of the vessel 100, thereby creating turbulence and enhancing the mixing that is effected by the mixing wheel 200b.

The size and shape of the deflector vanes 280b, 282b can be varied to vary the amount of axial flow generated thereby.

The omission of radial inflow elements, such as radial deflectors 260, 270, 272, 274 in the mixing wheel of 200b, has the benefit of simplifying the wheel manufacturing process, which can be especially advantageous for small scale bioreactors (e.g. below 50 liters) in which injection molding may be used for plastic wheel fabrication.

Thus, exemplary embodiments have been fully described above with reference to the drawing figures. Although the invention has been described based upon these exemplary embodiments, it would be apparent to those of skill in the art that certain modifications, variations, and alternative constructions could be made to the described embodiments within the spirit and scope of the invention.

1. A bioreactor comprising:
a vessel constructed and arranged to contain a fluid medium;
at least one medium addition conduit configured for conveying liquid medium into said vessel;
at least one medium removal conduit configured for conveying liquid medium from said vessel;
a gas inlet conduit configured for introducing a gas into said vessel; and
a mixing wheel mounted within said vessel for rotation about a substantially horizontal axis of rotation for mixing fluids within said vessel, said mixing wheel comprising:
a hollow cylinder;
a plurality of gas entrapment elements positioned about the outer periphery of said cylinder and configured to capture at least a portion of the gas introduced into said vessel by said gas inlet conduit and cause said mixing wheel to rotate due to the buoyancy of the entrapped gas; and
one or more deflector vanes disposed within an interior portion of said hollow cylinder and constructed and arranged to direct fluid within said hollow cylinder in a generally axial direction with respect to said cylinder.

2. The bioreactor of claim 1, further comprising one or more radial inflow elements disposed at one or more locations about the periphery of said cylinder, each radial inflow element being constructed and arranged to generate a radial fluid inflow into the interior portion of said cylinder when said mixing wheel is rotating about the axis of rotation, and wherein said deflector vanes are configured to redirect at least a portion of the radial fluid inflow in the generally axial direction with respect to said cylinder.

3. The bioreactor of claim 2, wherein said radial inflow elements comprise radial deflector vanes disposed across openings formed in said cylindrical wall.

4. The bioreactor of claim 1, wherein each deflector vane includes a leading edge portion extending beyond an axial end of said cylinder.

5. The bioreactor of claim 1, wherein each gas entrapment element comprises a partially enclosed cup having an opening facing in a circumferential direction with respect to said cylinder.

6. The bioreactor of claim 1, wherein said mixing element comprises a wheel with one or more flaps disposed about the periphery of said wheel, and wherein each flap is movable between a closed position lying along the circumference of said wheel and an opened position extending radially outwardly from the periphery of said wheel.

7. The bioreactor of claim 6, wherein each flap has a generally flat shape.

8. The bioreactor of claim 1, further comprising a rotation measuring element constructed and arranged to measure a rate of rotation of said mixing element.

9. The bioreactor of claim 1, wherein said vessel comprises a rigid container.

10. The bioreactor of claim 9, wherein said rigid container comprises injection molded plastic.

11. The bioreactor of claim 10, wherein said plastic comprises polyethylene terepthalate glycol (PETG).

12. The bioreactor of claim 9, further comprising a rigid housing supporting said rigid container.

13. The bioreactor of claim 1, wherein said vessel comprises a flexible bag, and wherein said bioreactor further comprises a rigid housing supporting said flexible bag.

14. The bioreactor of claim 13, wherein said flexible bag is formed from plastic.

15. The bioreactor of claim 14, wherein said plastic comprises polyethylene or polyvinylidene fluoride (PVDF).

16. The bioreactor of claim 13, wherein said flexible bag comprises generally parallel front and rear panels, generally parallel side panels, and a curved bottom panel, and wherein said housing comprises a rigid curved panel conforming to the shape of said curved panel of said flexible bag.

17. The bioreactor of claim 16, wherein said curved bottom panel of said flexible bag has a generally semi-circular curvature curved about an axis that is parallel to the axis of rotation of said mixing element.

18. The bioreactor of claim 1, wherein said at least one medium addition conduit comprises one or more of a base addition conduit connected to a source of base material, an inoculum/seed addition conduit connected to a source of inoculum/seed material, and a nutrition feed medium addition conduit connected to a source of nutrition feed medium.

19. The bioreactor of claim 1, further comprising a filter on said gas inlet conduit.

20. The bioreactor of claim 1, further comprising a heating element constructed and arranged to apply heat to the contents of said vessel.

21. The bioreactor of claim 1, further comprising sensors for detecting one or more characteristics of the fluid medium contained within said vessel.

22. The bioreactor of claim 21, wherein said sensors comprise one or more of a temperature sensor, a dissolved oxygen sensor, and a pH sensor.

23. The bioreactor of claim 2, wherein said radial inflow element comprises a vane angled in a direction of rotation of said mixing element.

24. The bioreactor of claim 1, wherein said mixing element is formed from plastic.

25. The bioreactor of claim 24, wherein said plastic comprises polyvinylidene fluoride (PVDF).

26. A bioreactor system comprising two or more bioreactor apparatuses of increasing volumetric capacity, each bioreactor apparatus comprising:
a fluid containment vessel constructed and arranged to contain a fluid medium and having a transverse dimension;
at least one medium addition conduit configured for conveying liquid medium into said fluid containment vessel;
at least one medium removal conduit configured for conveying liquid medium from said fluid containment vessel; and
a mixing rotor rotatably disposed within said fluid containment vessel and having a transverse dimension,
wherein, as the volumetric capacity of the bioreactor apparatuses increases, a ratio of the transverse dimension of the mixing rotor to the transverse dimension of the fluid containment vessel decreases.

27. The bioreactor system of claim 26, wherein said fluid containment vessel of each bioreactor apparatus comprises a bottom panel and wherein said mixing rotor of each bioreactor apparatus is disposed within said fluid containment vessel to define a gap between a bottom portion of said mixing rotor said bottom panel, and wherein, as the volumetric capacity of the bioreactor apparatuses increases the size of said gap increases.

28. A method for cultivating eukaryotic cells or microbial organisms comprising:
A. adding an amount of culture medium to a fluid containment vessel constructed and arranged to contain a fluid medium and having a transverse dimension;
B. adding materials to the culture medium to promote growth of the microbial organisms;
C. injecting a gas into the culture medium contained within the fluid containment vessel and using the injected gas to rotate a mixing rotor rotatably disposed within the fluid containment vessel and having a transverse diameter;
D. transferring the culture medium to another fluid containment vessel wherein the fluid containment vessel to which the culture medium is transferred has a larger volumetric capacity and larger transverse dimension than the fluid containment vessel from which the culture medium was transferred;
E. adding materials to the culture medium, including materials to promote growth of the eukaryotic cells or microbial organisms, until the volumetric capacity of the fluid containment vessel to which the culture medium was transferred is reached;
F. injecting a gas into the culture medium contained within the fluid containment vessel and using the injected gas to rotate a mixing rotor rotatably disposed within the fluid containment vessel to which the fluid medium was transferred, wherein the mixing rotor has a transverse diameter and wherein a ratio of the transverse diameter of the mixing rotor to the transverse dimension the fluid containment vessel to which the culture medium was transferred is less than a ratio of the transverse diameter of the mixing rotor to the transverse dimension the fluid containment vessel from which the culture medium was transferred.

29. The method of claim 28, further comprising repeating steps E-F until a desired volume of culture medium having a desired concentration of eukaryotic cells or microbial organisms is achieved.
